(19) United States
(12) Patent Application Publication
(10) Pub. No.: US 2022/0054023 A1
(43) Pub. Date: Feb. 24, 2022

(54) INTEGRATED BIOSENSING SYSTEMS

(71) Applicant: ams AG, Premstaetten (AT)

(72) Inventors: Bernhard Gruber, Eindhoven (NL); Manfred Pauritsch, Eindhoven (NL); Martin Dedek, Eindhoven (NL)

(21) Appl. No.: 17/415,338

(22) PCT Filed: Nov. 25, 2019
(86) PCT No.: PCT/EP2019/082484

§ 371 Date: Jun. 17, 2021

Related U.S. Application Data
(60) Provisional application No. 62/784,029, filed on Dec. 21, 2018.

(51) Int. Cl. A61B 5/026; A61B 5/00

(52) U.S. Cl. CPC A61B 5/026; A61B 2560/0242; A61B 2560/0223; A61B 5/0075

(57) ABSTRACT

An integrated sensing system for characterizing blood flow in a subject includes a light source assembly including a light source configured to emit light of a particular wavelength. The integrated sensing system includes an integrated circuit electrically connected to the light source assembly. The integrated circuit includes a light detector assembly including multiple light detectors configured to detect light of the particular wavelength; and a correlator configured to determining a delay between optical signals detected by respective light detectors of the light detector assembly.

### BACKGROUND

Optical sensors can be used to carry out photoplethysmographic (PPG) measurements, e.g., for heart rate monitoring or heart rate variability.

### SUMMARY

In an aspect, an integrated sensing system for characterizing blood flow in a subject includes a light source assembly including a light source configured to emit light of a particular wavelength. The integrated sensing system includes an integrated circuit electrically connected to the light source assembly. The integrated circuit includes a light detector assembly including multiple light detectors configured to detect light of the particular wavelength; and a correlator configured to determining a delay between optical signals detected by respective light detectors of the light detector assembly.

Embodiments can include one or more of the following features.

The light source assembly is configured to emit multiple wavelengths of light. The light source assembly includes a broad spectrum light source. The light source assembly includes multiple light sources each configured to emit light of a different wavelength.

The light detector assembly includes a spectral sensor configured to detect light of multiple wavelengths. Each light detector of the light detector assembly is configured to detect light of a corresponding wavelength. The integrated circuit including a multiplexer connected to an output of the spectral sensor.

The integrated circuit including a sequencer configured to control one or more of an operating frequency of the light source assembly and a sampling frequency of the light detector assembly.

The integrated sensing system includes an integrated module, in which the light source assembly and the integrated circuit are integrated into the integrated module.

The light source assembly includes a light emitting diode (LED).

The light source assembly includes a vertical cavity surface-emitting laser (VCSEL).

The light detectors include photodiodes.

In an aspect, a mobile computing device includes a sensing system having any one or more of the preceding features.

In an aspect, a vehicle includes a sensing system having any one or more of the preceding features.

In an aspect, a method for characterizing blood flow in a subject includes illuminating a blood vessel of the subject with light from a light source assembly electrically connected an integrated circuit. The method includes detecting, by a first light detector of a light detector assembly of the integrated circuit, a first optical signal indicative of a blood flow event at a first location in the blood vessel; and detecting, by a second light detector of the light detector assembly of the integrated circuit, a second optical signal indicative of a blood flow event at a second location in the blood vessel. The method includes, based on (i) a time delay between the first optical signal and the second optical signal and (ii) a separation between the first light detector and the second light detector, determining a characteristic of blood flow in the subject.

Embodiments can include one or more of the following features.

Determining a characteristic of blood flow in the subject includes determining a pulse transit time (PTT) of the subject.

The method includes determining the time delay between the first optical signal and the second optical signal. The method includes determining the time delay by a correlator of the integrated circuit.

The method includes controlling an operating frequency of the light source assembly by a sequencer of the integrated circuit.

The method includes controlling a sampling frequency of the first and second light detectors by a sequencer of the integrated circuit.

The light detector assembly includes a spectral sensor including multiple channels of operation. The method includes selecting a channel of operation for the spectral sensor. The method includes selecting a channel of operation for the spectral sensor based on a physical characteristic of the subject. The method includes selecting the channel of operation based on a skin tone of the subject. The method includes detecting the skin tone of the subject by detecting, by the spectral sensor, an absorption spectrum of the skin of the subject. The method includes controlling operation of the light source assembly based on the selected channel of operation for the spectral sensor.

The light detector assembly includes a spectral sensor including multiple channels of operation. The method includes detecting, by the spectral sensor, an optical characteristic of ambient light.

In an aspect, a method for determining a physical characteristic of a subject includes illuminating skin of the subject with light of multiple wavelengths from a light source assembly electrically connected to an integrated circuit; detecting, by a spectral sensor of the integrated circuit, a spectrum of light absorption by the skin; and determining a physical characteristic of the subject based on the detected spectrum of light absorption by the skin.

Embodiments can include one or more of the following features.

Determining a physical characteristic of the subject includes determining a skin tone of the subject.

The method includes determining the physical characteristic of the subject based on a comparison between the detected spectrum of light absorption by the skin and a reference spectrum.

Determining a physical characteristic of the subject includes determining an amount of beta carotene in the skin of the subject.

The integrated sensor systems described here can have one or more of the following advantages. The integrated sensor systems are compact and compatible with space constrained applications, such as mobile computing devices, e.g., wearable technology. The integrated sensor systems are easy to use and can implement optical measurement techniques such that they can be operated without contacting a subject. The optical measurements enabled by the integrated sensor systems can provide precise and accurate indications of blood flow characteristics such as blood pressure. The ability to select one or more channels for operation, e.g., dynamically based on ambient lighting or skin tone, enables a high signal-to-noise ratio to be achieved.

### BRIEF DESCRIPTION OF DRAWINGS

FIG. 1 is a diagram of an integrated sensing system.

FIGS. 2A and 2B are diagrams of integrated sensing systems.

FIG. 3 is a diagram of operation of an integrated sensing system.

FIG. 4 is a layout of an integrated circuit for an integrated sensing system.

FIGS. 5 and 6 are flow charts.

### DETAILED DESCRIPTION

We describe here integrated sensing systems for characterization of blood flow in a subject. For instance, the integrated sensing systems can be used to determine the pulse transit time of the subject, which is an indication of the subject's blood pressure. An integrated sensing system includes light sources and light detectors integrated into an integrated circuit, e.g., for incorporation into a mobile computing device, a steering wheel, or another device. The operation of the light sources and light detectors is synchronized by use of a correlator that correlates signals detected by multiple detectors, e.g., enabling time-dependent characterizations of blood flow, such as pulse wave velocity and pulse transit time, to be carried out. The integrated sensing systems can implement multiple illumination channels and multiple detection channels, e.g., to enable a high signal-to-noise ratio to be achieved and to enable sensing to be tuned to a particular subject or to a particular environment. The integrated sensing systems can also implement spectral analysis, e.g., for characterization of skin tone, identification of compounds such as beta carotene in a subject's tissue, or analysis of ambient lighting or color.

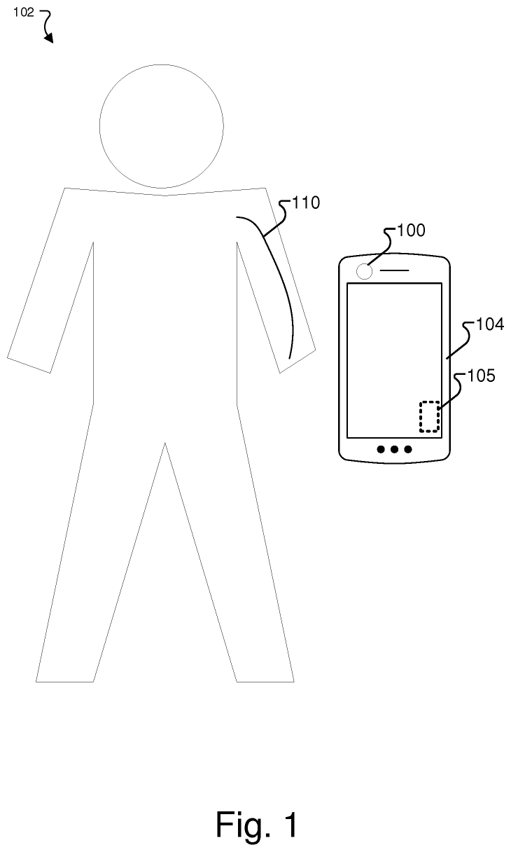

Referring to FIG. 1, an integrated sensing system 100 for characterization of blood flow in a subject 102 can be incorporated into a mobile computing device 104, e.g., a smartphone, tablet, wearable computing device, or other type of mobile computing device. In the example of FIG. 1, the mobile computing device 104 comprises a mobile phone. In some examples, the integrated sensing system 100 can be incorporated into another type of device, such as the steering wheel of a vehicle.

The integrated sensing system 100 can carry out photoplethysmography (PPG) measurements using a light source assembly and a light detector assembly of the integrated sensing system. When placed proximate to tissue of the subject 102, the light source assembly illuminates the tissue, such as a blood vessel 110 in the tissue, and the light detector assembly measures the light reflected or scattered by the tissue. Changes in the volume of blood passing through the blood vessel 110, e.g., caused by the systolic and diastolic phases of blood flow, cause changes to the light reflected or scattered by the blood vessel, such as changes to the intensity of the scattered or reflected light.

Changes in the reflected or scattered light caused by blood flow through the blood vessel 110 can be used to characterize blood flow in the subject 102. For instance, the optical signals detected by the light detector assembly can be analyzed to estimate or determine blood flow characteristics such as pulse wave velocity, blood pressure, pulse rate, pulse volume, peripheral arterial stiffness, blood oxygenation (e.g., SpO₂ levels), cardiac output, or other blood flow characteristics.

In some examples, the light source assembly can include multiple, distinct light sources. For instance, the light source assembly of the integrated sensing system 100 can emit multiple wavelengths of light, such as a broad spectrum of light (e.g., white light) or multiple discrete spectra. In some examples, the light detector assembly can include multiple, distinct light detectors. For instance, the light detector assembly can be a spectral sensor capable of detecting a spectrum of light, e.g., detecting a spectrum of the light reflected or scattered by the tissue. The availability of multiple channels of operation for the integrated sensor system 100 can enable a high signal-to noise ratio to be achieved, as discussed below, and can enable the operation of the integrated sensor system 100 to be tuned based on factors such as skin tone or ambient color or lighting.

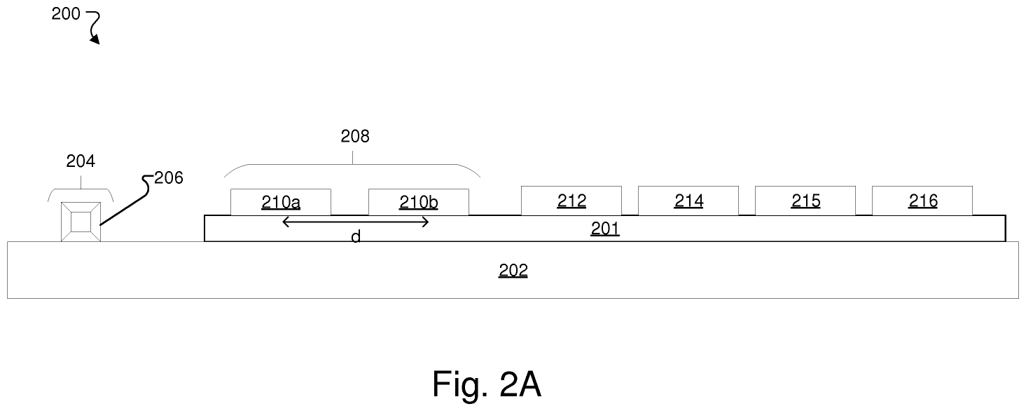

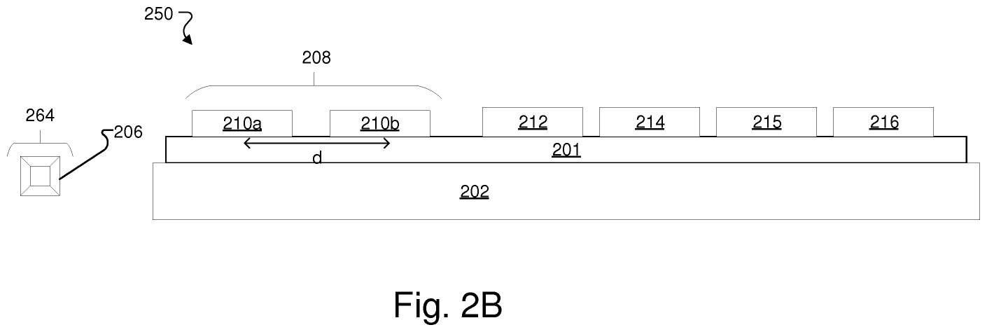

Referring to FIG. 2A, the integrated sensing system 100 includes an integrated module 200. In some examples, such as shown in FIG. 2A, the integrated module 200 includes a printed circuit board substrate 202, with components of the integrated sensing system 100 being electrically connected to conductive features of the printed circuit board. For instance, the components of the integrated sensing system can be connected to the printed circuit board substrate 202 by wire bonding, through-silicon vias, a backside redistribution layer, and connection elements such as solder balls. In some examples, the integrated module 200 can include a semiconductor chip 201, such as a silicon-based integrated circuit, having components of the integrated sensing system 100 formed in the chip, e.g., using semiconductor processing techniques.

A light source assembly 204 is integrated into the integrated module 200 of the integrated sensing system 100. The light source assembly 204 can include one or more light sources, such as light emitting diodes (LEDs), semiconductor lasers such as vertical cavity surface emitting lasers (VCSELs), or other types of light sources. In the example shown, the light source assembly 204 includes a single light source 206. In some examples, the light source assembly 204 can include multiple light sources.

The light source assembly 204 can emit multiple wavelengths of light, e.g., infrared light, visible light, or ultraviolet light, or a combination thereof. For instance, when the light source assembly 204 includes a single light source 206, the light source can be a broad spectrum light source configured to emit light in a broad wavelength range, e.g., a white LED. When the light source assembly 204 includes multiple light sources, each light source can be a broad spectrum light source, or each light source can emit light of a different spectrum (e.g., light in a different wavelength range).

The light detector assembly 208 is integrated into the integrated module 200 of the integrated sensing system 100, e.g., formed in the semiconductor chip 201. The light detector assembly 208 can include one or more light detectors, such as photodiodes. In the example shown, the light detector assembly 208 includes multiple light detectors 210a, 210b (sometimes referred to collectively as light detectors 210). In some examples, the light detector assembly 208 can include only a single light detector, or can include more than two light detectors. The light detector assembly 208 is configured to detect light of at least one of the wavelengths of the light emitted by the light source assembly 204. In some examples, the light detector assembly 208 can be a spectral sensor having multiple channels for detection of light at multiple wavelengths. For instance, a spectral sensor light detector assembly 208 can include multiple light detectors each configured to detect light in a corresponding wavelength range. The light detector assembly 208 can be configured to detect infrared light, visible light, or ultraviolet light, or a combination thereof

A change in blood volume in the blood vessel of a subject, e.g., the propagation of a blood pressure wave through the blood vessel, can cause a change in the intensity of light reflected or scattered by the blood vessel, which results in a change in the PPG signal detected by the light detector assembly. The intensity of the reflected or scattered light over time, as detected by the light detector assembly 208, can be used to characterize the blood flow. For instance, a PPG signal has a constant component that depends on the reflection or scattering of light due to tissue, and a periodically changing component caused by the propagation of a blood pressure wave through the blood vessel. Features such as the intensity and time constant of the periodically changing component of the PPG signal can be used for blood flow characterization.

In some examples, the integrated sensing system 100 can take measurements that can be used to determine a pulse wave velocity of a blood pressure wave in a blood vessel of a subject. The pulse wave velocity can then be used to determine a pulse transit time (PTT) for the subject, which is the amount of time it takes a PPG wave to travel between two sites and is an indicator of the subject's pulse wave velocity (PWV).

To carry out pulse wave velocity measurements, the integrated sensing system 100 can have multiple light detectors 210, multiple light sources 206, or both. The following discussion refers to the example of FIG. 2A, in which the light detector assembly 208 includes multiple light detectors 210a, 210b. A similar approach can be taken for the case in which the light source assembly 204 includes multiple light sources.

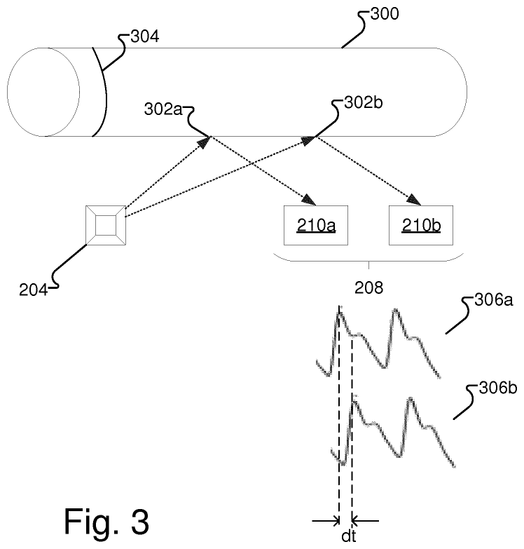

Referring also to FIG. 3, a blood vessel 300 of a subject is illuminated with light from the light source assembly 204 of the integrated sensing system 100. The two light detectors 210a, 210b of the light detector assembly 208 are separated by a distance d such that each light detector 210a, 210b detects light reflected or scattered from a corresponding point 302a, 302b along the blood vessel 300. The distance d can be less than 10 mm, e.g., between 2 mm and 10 mm. As blood pressure waves 304 propagate along the blood vessel 300, they arrive first at the first point 302a, and subsequently at the second point 302b. This time delay between the arrival of the blood pressure wave 304 at the first point 302a and the arrival of the blood pressure wave 304 at the second point 302b means that the light detector 210a detects a PPG signal 306a that is shifted in time by an amount dt relative to the PPG signal 306b detected by the light detector 210b.

Based on the time delay between the arrival of the blood pressure wave 304 at the first point 302a and the arrival of the blood pressure wave 304 at the second point 302b and on the separation d between the light detectors 210a, 210b, the pulse wave velocity in the blood vessel 300 can be determined. From the pulse wave velocity, the PTT and the blood pressure can also be determined.

Referring again to FIG. 2A, a correlator 212 integrated into the integrated circuit 201 of the integrated sensing system 100 is electrically connected to an output of an analog-to-digital converter 215 that receives analog signals from the light detectors 210a, 210b. The correlator 212 is operable to correlate the PPG signals 306a, 306b detected by the light detectors 210a, 210b such that the time delay can be determined. The correlation of the PPG signals 306a, 306b can also reduce the noise in the PPG signals, improving the signal-to-noise ratio.

In some examples, a sequencer 214 can be integrated into the integrated circuit 201 of the integrated sensing system 100. The sequencer 214 can control the frequency of the operation of the light source assembly 204, the sampling frequency of the light detector assembly 208, or both. For instance, the operation of the sequencer 214, and the subsequent correlation of the PPG signals 306a, 306b, can enable coherent sampling by the light detectors 210a, 210b. In some examples, the light source assembly 204 or the light detector assembly or both can be operated continuously. In some examples, the sequencer 214 can control the light source assembly 204 or the light detector assembly or both to operate at a frequency of, e.g., between 100 Hz and 500 Hz.

Signals from the integrated sensor system 100, such as PPG signals detected by the light detector assembly 208, correlation output from the correlator 212, or other signals, can be processed to determine blood flow characteristics of the subject. For example, as discussed above, pulse wave velocity, pulse transit time, and blood pressure can be determined based on signals from the integrated sensor system 100. In some examples, other characteristics of blood flow can also be determined by the signals from the integrated sensor system 100, such as pulse rate, pulse volume, peripheral arterial stiffness, blood oxygenation (e.g., SpO₂ levels), cardiac output, or other blood flow characteristics.

In some examples, the signal processing can be carried out by one or more processors integrated into the integrated sensor system 100, e.g., a processor integrated into the integrated circuit 201 or a processor that is part of the integrated module 200. In some examples (e.g., as shown in FIG. 1), one or more processors 105 of the device 104 in which the integrated sensor system 100 is integrated can carry out the signal processing. For instance, in the example of FIG. 1, the one or more processors 105 of the mobile phone can perform the signal processing operations to determine blood flow characteristics based on the output signals from the integrated sensor system 100.

In some examples, e.g., when the light detector assembly 208 includes a spectral sensor having multiple channels, the integrated sensing system can perform spectroscopy. For instance, the light source assembly 204 (e.g., a broad spectrum light source or multiple discrete light sources) can illuminate tissue at multiple wavelengths, e.g., by activating a broadband light source such as a white LED, and the spectral sensor light detector assembly 208 can detect the scattered or reflected light intensity in multiple channels, each channel corresponding to a wavelength range. In these examples, the output of the spectral sensor light detector assembly 208 is a spectrum of the scattered or reflected light.

The spectrum of scattered or reflected light detected by the spectral sensor light detector assembly 208 can be indicative of a skin tone of the subject. In some examples, the operation of the integrated sensor system 100 for characterizing blood flow in the subject can be controlled based on the skin tone of the subject, as indicated by the spectrum of scattered or reflected light. For instance, a skin tone that absorbs strongly in the red may give a low signal-to-noise ratio if illuminated with red light, so the sensor system can be controlled to illuminate with, and detect, green or blue light. In some examples, darker skin tones can be generally more absorbent, and so the power of the light source assembly can be increased.

In some examples, a multiplexer 216 integrated into the integrated circuit 201 of the integrated sensor system 100 can control the channel of operation of the light detector assembly 208, e.g., based on the detected skin tone of the subject. For instance, for a subject with a reddish skin tone, the multiplexer 216 can enable channels in the green and blue, where the intensity of the light scattered from the skin of the subject is higher. This dynamic selection of channels of the light detector assembly 208 can help to improve the signal-to-noise ratio of the PPG signal output from the light detector assembly 208. In some examples, another multiplexer (not shown) can control the operation of the light source assembly 204, e.g., by enabling a light source of a certain color. Illuminating the subject only with light of certain wavelengths can help reduce power consumption of the integrated sensor system 100.

In some examples, the spectrum of scattered or reflected light can be indicative of the presence of a compound in the tissue of the subject. For instance, beta carotene absorbs strongly in the red. Based on the intensity of the scattered or reflected light in the red as compared to the intensity of the scattered or reflected light elsewhere in the spectrum, a qualitative or quantitative indication of the amount of beta carotene present in the subject's tissue can be determined. The presence of beta carotene is an indicator of health: health issues such as stress or the presence of free radicals can reduce the concentration of beta carotene. The spectrum of scattered or reflected light can be interpreted as a general indication of the subject's health.

In some examples, the integrated sensor system 100 can be used to detect an ambient color, such as a color of lighting in a room. For instance, the light detector assembly 208 can be operated to detect a spectrum of ambient light without activation of the light source assembly 204. In some examples, it may be desirable to control the channel of operation of the light detector assembly 208, the operation of the light source assembly 204, or both, based on the ambient color, e.g., for PPG measurements.

In some examples, the channel of operation of the light detector assembly 208 can be controlled based on an environmental criterion. For instance, hemoglobin absorbs green light better than red and infrared light, resulting in a stronger PPG signal for green light than for red light. As blood flow changes, e.g., as a blood pressure wave propagates along a blood vessel, there is an accordingly larger change in the reflected light when green light is used than when red light is used, resulting in a better signal-to-noise ratio. However, infrared illumination can penetrate deeper into the skin, so can reach blood vessels deep in the skin, where green light interacts primarily with blood vessels at the surface of the skin. In some cases, such as in cold environments where a subject has decreased microcirculation, infrared illumination may be preferable to green illumination. The channel of operation of the light detector assembly 208 can be controlled to account for such environmental factors.

The detected ambient color can be used in other contexts. For instance, the color balance or lighting balance of a camera of the mobile computing device 104 in which the integrated sensor system 100 is integrated can be adjusted based on the spectrum of ambient light in a room.

In some examples, the integrated sensor system 100 can be used to identify a color of an object, such as the color of paint on a wall or the color of a fabric. For instance, the light detector assembly 208 can be operated to detect a spectrum of light reflected or scattered by an object without activation of the light detector assembly 208.

In some examples, a capacitive electrocardiogram (ECG) module can be integrated into the integrated circuit of the integrated sensor system 100. The ECG module can be coupled to electrodes that measure an electrical signal from the tissue indicative of cardiac function. The operation of the ECG module can be controlled by the sequencer 214. For instance, the sequencer 214 can control the sampling frequency of the ECG module. The sampling frequency of the ECG module can be controlled independently from the sampling frequency of the light detector assembly 208, e.g., enabling both the ECG module and the light detector assembly 208 to be operated at an appropriate sampling frequency. For instance, ECG, which detects sudden and rapid spikes in an electrical signal, can be operated at a higher sampling frequency than the light detector assembly 208, which detects a generally smoothly varying optical signal.

Referring to FIG. 2B, in some examples, an integrated module 250 of the integrated sensing system 100 can include the semiconductor chip 201, e.g., mounted on printed circuit board substrate 202, as described above with respect to FIG. 2A. A light source assembly 264 including one or more light sources is formed separately from the integrated module 250.

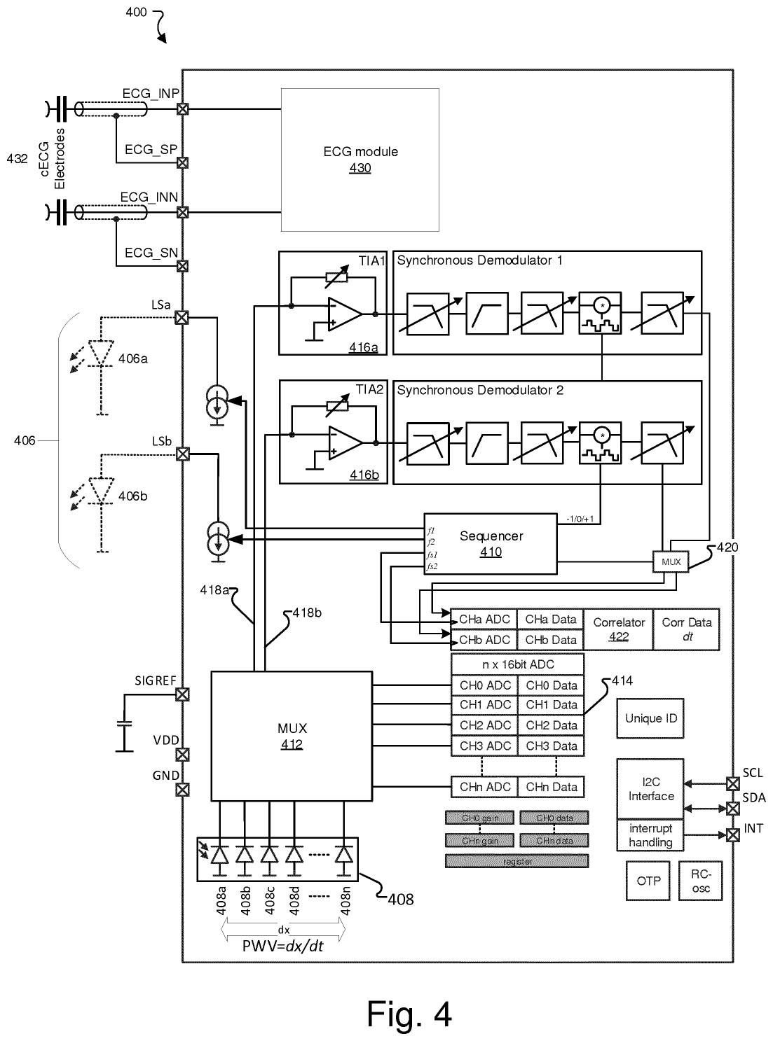

FIG. 4 shows an example circuit layout for an integrated circuit 400 of an integrated module of an integrated sensing system, such as that described above. The integrated circuit 400 includes a light source assembly 406 with two light sources 406a, 406b. In some examples, the light source assembly 406 can be part of the integrated module (e.g., as shown in FIG. 2A), or can be external to and not integrated with the integrated module (as shown in FIG. 2B). A sequencer 410 is connected to the light source assembly 406 to control the operation, e.g., the frequency of operation, of the light source assembly 406.

The integrated circuit 400 also include a light detector assembly 408 with multiple light detectors 408a-408n. The distance between the first light detector 408a and the last light detector 408b is a fixed distance dx. The outputs of the light detector assembly 408 are connected to a multiplexer 412 that can enable one or more channels of the light detector assembly 408, e.g., based on a desired wavelength for detection of the PPG signals. For instance, for pulse wave velocity determination, the multiplexer 412 enables two of the channels of the light detector assembly 408.

The output from the multiplexer 412 is connected to a pair of impedance amplifiers 416a, 416b (collectively referred to as impedance amplifiers 416), each of which defines a corresponding signal path 418a, 418b for the output from a corresponding one of the enabled channels of the light detector assembly 408. The impedance amplifiers 416 provide sufficient impedance to the light detector assembly 408 circuitry such that the light detectors can operate with a sampling frequency sufficient to obtain a PPG signal with enough resolution to determine characteristics such as pulse wave velocity and pulse transit time.

The signal paths 418 are fed into a multiplexer 420 that is driven by the sequencer 410 and then into a correlator 422. The output of the correlator 422 is the time shift between the signals on the two enabled channels of the light detector assembly 408. By integrating the correlator 422 into the integrated circuit 400 itself, the correlation of signals and the determination of the time shift can be performed directly on the chip, which can be faster and more efficient than having such processes performed by an external processor.

The light detector 408 assembly is connected to a multi-channel analog-to-digital converter (ADC) 414 via the multiplexer 412. For instance, the ADC 414 can be a light-to-frequency (LTF) converter that converts current into digital data. The multiplexer 414 can enable one or more of the channels of the light detector assembly 408 to be provided to the ADC 414, with the output from the ADC 414 being provided to one or more processors for signal processing analysis. For instance, output from the ADC 414 can be for spectral analysis, e.g., for skin tone determination, ambient color determination, identification of compounds such as beta carotene in the tissue, or other types of spectral analysis.

In some examples, concurrent measurement of pulse wave velocity and collection of an intensity spectrum can be performed by the integrated circuit 400. The sequencer 410 can control the multiplexer 412 to switch from the connection to the correlator 422 to the connection to the ADC 414. For instance, when the light source assembly 406 is switched on, the sequencer can first control the multiplexer 412 to enable the connection to the correlator. After a sufficient amount of sampling time has elapsed, and while the light source assembly 406 is still switched on, the sequencer can control the multiplexer 412 to enable the connection to the ADC 414, e.g., for skin tone or ambient light characterization.

In some examples, the integrated circuit 400 includes an ECG module 430 for measuring an electrical signal, such as an ECG signal. The ECG module 430 can include electrodes 432 that can be placed in electrical contact with the tissue of a subject.

In some examples, the operation of the integrated circuit 400 is controlled by a controller 440, such as an I2C interface, e.g., an I2C interface, SPI interface, or another type of interface. In some examples, the operation of the integrated circuit 400 is controlled by a controller of the device in which the integrated sensing system is integrated, such as a controller of the mobile computing device.

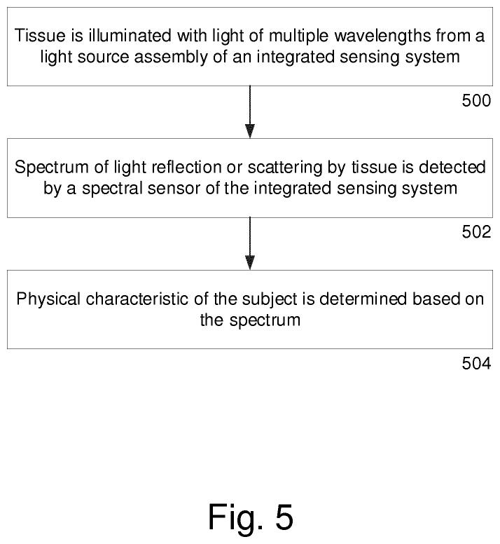

Referring to FIG. 5, in an example process for determining a physical characteristic of a subject, tissue of the subject is illuminated with light of multiple wavelengths from a light source assembly of an integrated sensing system (500). The subject's tissue reflects and/or scatters the light, and a spectrum of light reflection or scattering is detected by a spectral sensor of the integrated sensing system (502). A physical characteristic of the subject is determined based on the spectrum of light reflection or scattering by the subject's tissue (504). For instance, the subject's skin tone can be determined, or the presence of a compound can be identified, e.g., based on a comparison between the detected spectrum of light absorption and a reference spectrum.

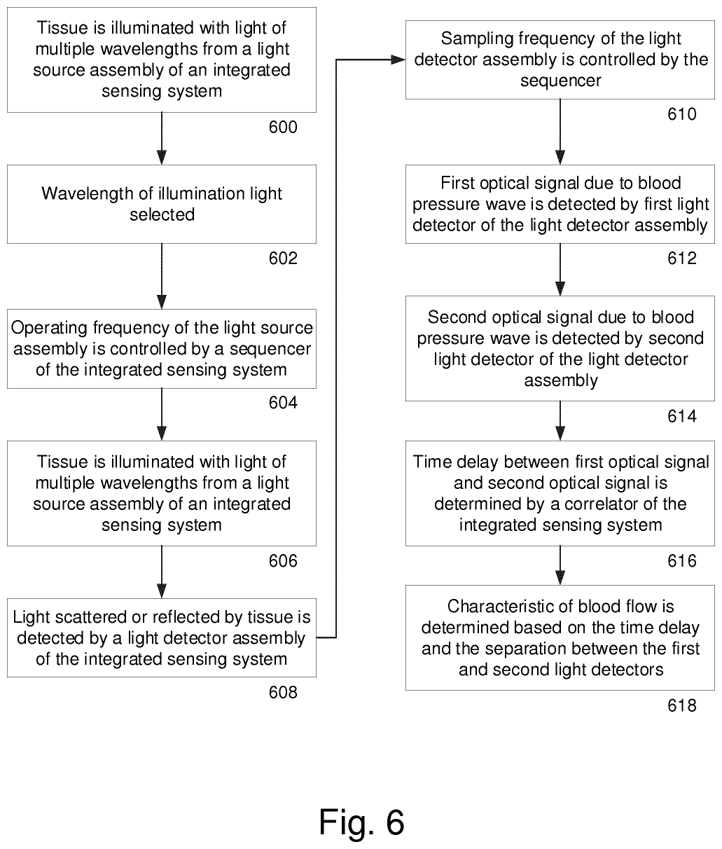

Referring to FIG. 6, in an example process for characterizing blood flow in a subject, a blood vessel of the subject is illuminated with light from a light source assembly of an integrated sensing system (600). In some examples, the wavelength of the illumination light can be selected (602), e.g., based on a skin tone or other characteristic of the subject. In some examples, an operating frequency of the light source assembly is controlled by a sequencer of the integrated sensing system (604). The tissue of the subject, including the blood vessel, scatters or reflects the light, and the scattered or reflected light is detected by light detectors of a light detector assembly (606). In some examples, a sampling frequency of the light detector assembly is controlled by the sequencer (608). When a blood pressure wave passes by a first point along the length of the blood vessel, it causes a change in the light scattering or reflection by that point, and a first optical signal is detected by a first one of the light detectors (610), the optical signal indicative of the blood pressure wave changing the light scattering. The blood pressure wave then passes by a second point along the length of the blood vessel, causing a change in the light scattering by that second point, and a second optical signal is detected by a second one of the light detectors (612). A time delay between detection of the first optical signal by the first light detector and detection of the second optical signal by the second light detector is determined by a correlator of the integrated sensing system (614). A characteristic of the blood flow, such as a pulse wave velocity, is determined based on the time delay and the separation distance between the two light detectors (616). In some examples, further characteristics of the subject's blood flow can then be determined. For instance, a pulse transit time can be determined based on the pulse wave velocity, and the subject's blood pressure can be determined or estimated based on the pulse transit time or based directly on the pulse wave velocity (618).

A number of embodiments have been described. Nevertheless, it will be understood that various modifications may be made without departing from the spirit and scope of the invention. For example, some of the steps described above may be order independent, and thus can be performed in an order different from that described.

Other implementations are also within the scope of the following claims.

1-30. (canceled)

31. An integrated sensing system for characterizing blood flow in a subject, the sensing system comprising:
a light source assembly comprising a light source configured to emit light of a particular wavelength; and
an integrated circuit electrically connected to the light source assembly, the integrated circuit comprising;
a light detector assembly comprising multiple light detectors configured to detect light of the particular wavelength;
a correlator configured to determining a delay between optical signals detected by respective light detectors of the light detector assembly.

32. The integrated sensing system of claim 31, in which the light source assembly is configured to emit multiple wavelengths of light; optionally
in which the light source assembly comprises a broad spectrum light source; and/or
in which the light source assembly comprises multiple light sources each configured to emit light of a different wavelength.

33. The integrated sensing system of claim 31, in which the light detector assembly comprises a spectral sensor configured to detect light of multiple wavelengths; optionally
in which each light detector of the light detector assembly is configured to detect light of a corresponding wavelength.

34. The integrated sensing system of claim 33, the integrated circuit comprising a multiplexer connected to an output of the spectral sensor.

35. The integrated sensing system of claim 31, the integrated circuit comprising a sequencer configured to control one or more of an operating frequency of the light source assembly and a sampling frequency of the light detector assembly.

36. The integrated sensing system of claim 31, comprising an integrated module, in which the light source assembly and the integrated circuit are integrated into the integrated module.

37. The integrated sensing system of claim 31, in which the light source assembly comprises a light emitting diode (LED) and/or a vertical cavity surface-emitting laser (VCSEL).

38. The integrated sensing system of claim 31, in which the light detectors comprise photodiodes.

39. A mobile computing device comprising the sensing system of claim 31.

40. A vehicle comprising the sensing system of claim 31.

41. A method for characterizing blood flow in a subject, the method comprising:
illuminating a blood vessel of the subject with light from a light source assembly electrically connected an integrated circuit;
detecting, by a first light detector of a light detector assembly of the integrated circuit, a first optical signal indicative of a blood flow event at a first location in the blood vessel;
detecting, by a second light detector of the light detector assembly of the integrated circuit, a second optical signal indicative of a blood flow event at a second location in the blood vessel;
based on (i) a time delay between the first optical signal and the second optical signal and (ii) a separation between the first light detector and the second light detector, determining a characteristic of blood flow in the subject.

42. The method of claim 41, in which determining a characteristic of blood flow in the subject comprises determining a pulse transit time (PTT) of the subject.

43. The method of claim 41, comprising determining the time delay between the first optical signal and the second optical signal; optionally
comprising determining the time delay by a correlator of the integrated circuit.

44. The method of claim 41, comprising controlling an operating frequency of the light source assembly by a sequencer of the integrated circuit; and/or
comprising controlling a sampling frequency of the first and second light detectors by a sequencer of the integrated circuit.

45. The method of claim 41, in which the light detector assembly comprises a spectral sensor comprising multiple channels of operation, and
the method comprising selecting a channel of operation for the spectral sensor; optionally
further comprising controlling operation of the light source assembly based on the selected channel of operation for the spectral sensor.

46. The method of claim 45, comprising selecting a channel of operation for the spectral sensor based on a physical characteristic of the subject; optionally
comprising selecting the channel of operation based on a skin tone of the subject; further optionally
comprising detecting the skin tone of the subject by detecting, by the spectral sensor, an absorption spectrum of the skin of the subject.

47. The method of claim 41, in which the light detector assembly comprises a spectral sensor comprising multiple channels of operation, and
the method comprising detecting, by the spectral sensor, an optical characteristic of ambient light.

48. A method for determining a physical characteristic of a subject, the method comprising:
illuminating skin of the subject with light of multiple wavelengths from a light source assembly electrically connected to an integrated circuit;
detecting, by a spectral sensor of the integrated circuit, a spectrum of light absorption by the skin; and
determining a physical characteristic of the subject based on the detected spectrum of light absorption by the skin.

49. The method of claim 48, in which determining a physical characteristic of the subject comprises determining a skin tone of the subject; and/or
comprising determining the physical characteristic of the subject based on a comparison between the detected spectrum of light absorption by the skin and a reference spectrum.

50. The method of claim 48 in which determining a physical characteristic of the subject comprises determining an amount of beta carotene in the skin of the subject.
